(19) United States
(12) Patent Application Publication
(10) Pub. No.: US 2012/0029255 A1
(43) Pub. Date: Feb. 2, 2012

(54) PROCESS FOR CONVERTING A HEAVY FEED INTO GASOLINE AND PROPYLENE, HAVING AN ADJUSTABLE YIELD STRUCTURE

(75) Inventors: Romina Digne, Lyon (FR); Olivier Callebert, Nanterre (FR); Romain Roux, Rueil Malmaison (FR)

(73) Assignee: IFP Energies nouvelles, Rueil-Malmaison Cedex (FR)

(21) Appl. No.: 13/060,457

(22) PCT Filed: Jul. 29, 2009
(86) PCT No.: PCT/FR09/00945

§ 371 Date: Oct. 14, 2011

(30) Foreign Application Priority Data
Aug. 29, 2008 (FR) 08/04759

(51) Int. Cl. C07C 4/06

(52) U.S. Cl. 585/324

(57) ABSTRACT

The present invention describes a process for conversion of a heavy feed, having high flexibility as regards the production of propylene and gasoline. The process uses a catalytic cracking unit followed by a unit for oligomerization of C3/C4 olefins, C4 or C4/C5 olefins depending on the selected production. The process of the invention encompasses cases in which catalytic cracking is carried out in a single reactor or in two distinct reactors, each reactor possibly functioning in riser or dropper mode.

Optionally, an olefinic cut hydrogenation unit may be placed upstream of the oligomerization unit.

### FIELD OF THE INVENTION

The invention relates to a process for conversion of a heavy hydrocarbon feed, having high flexibility regarding the production of gasoline and propylene. More precisely, the process of the present invention can co-produce gasoline in a minimum yield and propylene with a yield which may reach 10% of the weight of the feed.

The minimum yield of gasoline depends on the starting feed, but routine feeds such as vacuum distillates or atmospheric residues, this minimum yield is more than 40%, and preferably more than 45% by weight with respect to the entering feed.

Demand for propylene has been increasing strongly for many years. The principal source of propylene production is the steam cracking of naphtha. Since the increase in propylene yield from the steam cracking process is limited, refiners have been prompted to produce more and more propylene using the catalytic cracking process known as FCC, fluid catalytic cracking.

The FCC process can convert heavy hydrocarbon feeds the initial boiling point of which is generally more than 340° C. to lighter hydrocarbon fractions, in particular a gasoline cut, by cracking molecules of the heavy feed in the presence of an acid catalyst.

FCC also produces liquefied petroleum gas (LPG) in large quantities with high olefins contents.

Carrying out catalytic cracking orientated towards the production of propylene necessitates either using more severe operating conditions (increase the temperature at the FCC reactor outlet and moving the catalyst faster) or using specific additives for the cracking catalyst or a combination of those two principal means.

In general, the propylene yield is obtained to the detriment of the gasoline yield and is associated with relatively severe operating conditions.

In the process of the present invention, the gasoline yield is maintained at a minimum value which clearly depends on the nature of the feed and the catalyst used, even when the operating conditions are determined with a view to maximizing the propylene production.

When propylene production is not the intention, the process can then be allowed to maximize gasoline production.

The process of the present invention can thus generally be presented as a process for the co-production of propylene and gasoline with a minimum gasoline yield.

The process employs, in succession, the reactions of catalytic cracking and oligomerization of olefins from the C3/C4 cut, or from the C4 cut, or from the C4/C5 cut, the term Cn denoting a cut of hydrocarbons containing n carbon atoms. This process produces gasoline with a minimum yield which is generally more than 40% by weight with respect to the entering feed and propylene in a yield which can be adjusted within a wide range, which for certain feeds can be up to 10% by weight.

The present invention is compatible with all catalytic cracking reactor technologies, whether using riser or dropper technology.

### EXAMINATION OF THE PRIOR ART

Patent FR-2 837 213 describes a process for conversion of a heavy feed which comprises a step for catalytic cracking, selective hydrogenation and oligomerization of olefins containing 4 and/or 5 carbon atoms derived from catalytic cracking. The heavy feed and the oligomers are cracked together or separately with the same catalyst. The cracking effluents are separated in a common fractionation zone. A portion of the C4 or C4/C5 cut obtained after fractionation is oligomerized. When the C4 cut is oligomerized then cracked as a secondary feed, this process increases the propylene yield while retaining the gasoline yield. In common with the cited patent, the process of the present invention can increase the propylene yield while slightly increasing the gasoline yield, but it offers in addition the possibility of substantially increasing the gasoline yield and the capacity of the unit if the propylene production is no longer desired, all using the same facility and without changing the catalyst.

Patent FR-2 837 100 describes a process for producing propylene and gasoline comprising at least one step for oligomerization and a step for catalytic cracking of the oligomers formed. The oligomers formed from olefins containing 4 and/or 5 carbon atoms are cracked in a catalytic cracking unit to form large quantities of propylene. The feed for the process of the cited patent is thus a “light” hydrocarbon feed with a boiling point of less than 340° C. In contrast to the process described in patent FR-2 837 199, the process of the present invention concerns the conversion of a heavy feed with a boiling point of more than 340° C. in order to obtain a lot of gasoline and more or less propylene.

### BRIEF DESCRIPTION OF THE FIGURES

FIG. 1 shows a flow chart for the process of the invention in its “maxi propylene” regime;

FIG. 2 shows a flow chart for the process of the invention in its “maxi gasoline” regime.

### BRIEF DESCRIPTION OF THE INVENTION

The invention concerns a process for conversion of a hydrocarbon feed termed a heavy feed, i.e. constituted by hydrocarbons with a boiling point of more than approximately 340° C., with a view to the co-production of propylene and gasoline with a minimum yield.

The process of the invention comprises at least two reaction steps, a first catalytic cracking step and a second step for oligomerization of C3 and C4 olefins, or C4 olefins or C4 and C5 olefins, derived from catalytic cracking.

A third reaction step for selective olefin hydrogenation may in certain cases be necessary before oligomerization.

The process of the invention means that two types of production corresponding to two distinct regimes can be carried out:
a regime termed “maxi propylene” corresponding to a maximum production of propylene while maintaining a minimum or even slightly increased gasoline yield with respect to the potential yield of the catalytic cracking unit alone; or
a regime termed “maxi gasoline”, corresponding to a maximum production of gasoline without the production of propylene.

One of the advantages of the invention is to be able to swing over time from one to the other of the two regimes defined above. It is also possible to operate the unit in any intermediate mode between the “maxi propylene” and “maxi gasoline” regimes.

This swing is very simple to carry out as it essentially consists of modifying the feed entering the oligomerization unit without substantially modifying the operating conditions of the catalytic cracking unit and the oligomerization unit and clearly without modifying the catalysts used.

### In “Maxi Propylene” Mode

the heavy feed is cracked in a fluidized bed catalytic cracking reactor in the presence of a cracking catalyst;
a second relatively light feed is cracked with the same cracking catalyst, separately or as a mixture with the heavy feed. This second feed comprises olefins containing at least 8 carbon atoms which have been produced by oligomerization of olefins containing 4 (or 4 and 5) carbon atoms derived from catalytic cracking. We shall henceforth refer to the C4 olefinic cut or the C4/C5 olefinic cut;
the cracking effluents from the two feeds are sent to a common fractionation zone and the catalyst used for cracking of the two feeds is regenerated in a common regeneration zone;
the C4 (or C4/C5) olefinic cut from catalytic cracking is sent to an oligomerization unit. Selective hydrogenation of the C4 (or C4/C5) cut before the oligomerization unit may be necessary, as well as a prior treatment, to remove impurities such as nitrogen-containing and sulphur-containing compounds. A portion of the oligomers formed are sent for catalytic cracking to produce propylene as they are good propylene precursors. The remainder of the non-recycled oligomers is mixed with the gasoline cut produced by the catalytic cracking.
   In “maxi gasoline mode”
the heavy feed is cracked in the catalytic cracking reactor in the presence of the same cracking catalyst as that used in the “maxi propylene” mode. Since there is no other cracked feed, a quantity of heavy feed which is greater than that of the maxi propylene mode may be converted;
the C3/C4 cut from catalytic cracking is sent to an oligomerization unit. Selective hydrogenation of the C3/C4 cut upstream of the oligomerization unit may be necessary;
the C6+ oligomers formed are separated into a gasoline cut which is mixed with the gasoline cut produced by catalytic cracking and a heavier cut which may be incorporated into the gas oil pool.

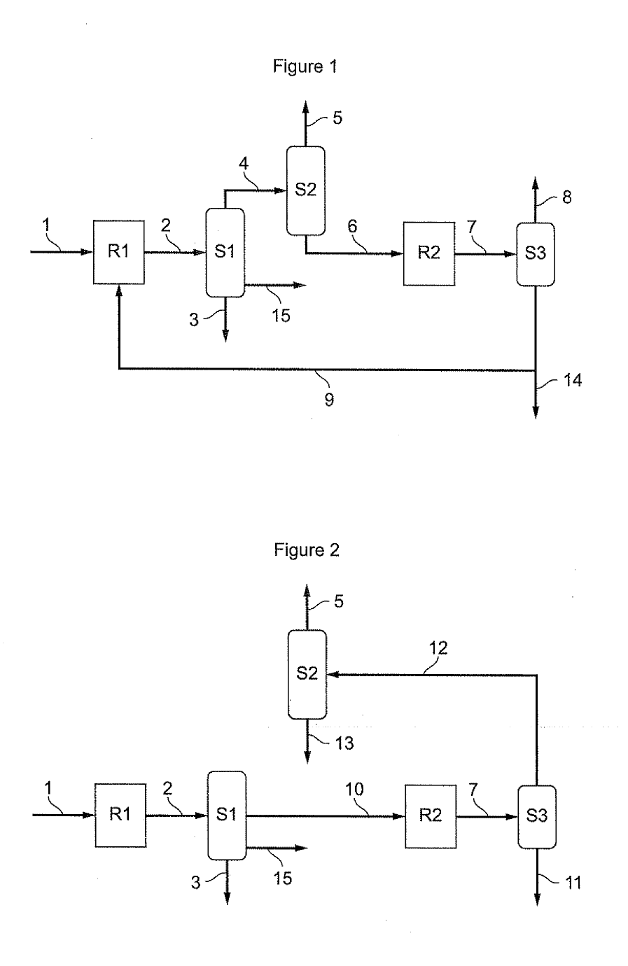

As can be seen in the flowcharts for the process (FIG. 1 for the maxi propylene regime, FIG. 2 for the maxi gasoline regime), swinging from one regime to the other is very simple. As an example, in order to pass from the “maxi propylene” regime to the “maxi gasoline” regime, it suffices to send the C3+C4 olefinic cut (and not just the C4 cut or the C4+C5 cut) to the oligomerization unit, no longer recycling the oligomers formed to the catalytic cracking unit.

More precisely, the process for co-production of gasoline and propylene from a hydrocarbon feed with an initial boiling point of more than 340° C. of the invention uses a catalytic cracking unit followed by an oligomerization unit which can function in accordance with two regimes termed “maxi propylene” (regime 1) and “maxi gasoline” (regime 2) in which:
for the “maxi propylene” regime, the feed for the oligomerization unit is constituted by the C4 cut or the C4/C5 olefinic cut derived from catalytic cracking and the effluents from the oligomerization unit are separated into a C8+ oligomer cut which is recycled at least in part to the inlet to the catalytic cracking unit, the gasoline cut from the process being constituted by the gasoline cut produced in the catalytic cracking unit and the oligomer cut derived from the oligomerization unit which is not recycled for catalytic cracking;
for the “maxi gasoline” regime, the feed for the oligomerization unit is constitute by the C3/C4 olefinic cut derived from catalytic cracking, and the effluents from the oligomerization unit are separated into a C6+ oligomer cut which is added to the gasoline cut derived from catalytic cracking to constitute the gasoline produced by the process;
   the propylene being obtained in the two regimes from FCC effluents following separation in one or more distillation columns.

As will be defined in the next paragraph, the catalytic cracking unit may be broken down into several modalities with a single reactor processing the heavy feed and the light feed or two reactors, one processing the heavy feed and the other processing the light feed. Further, each reactor may function in riser or dropper mode.

### DETAILED DESCRIPTION OF THE INVENTION

According to the invention, the overall feed to be cracked contains more than 50% by weight of hydrocarbons with a boiling point of more than 340° C. In general, the feed is constituted by a vacuum distillate or possibly an atmospheric residue. The overall cracked feed may contain up to 100% by weight of hydrocarbons with a boiling point of more than 340° C. for the “maxi gasoline” mode. For the “maxi propylene” mode, the feed usually contains more than 60% by weight, and more usually more than 70%, for example between 70% and 95% by weight of hydrocarbons with a boiling point of more than 340° C.

The secondary feed for catalytic cracking required in the “maxi propylene” mode generally contains 2% to 40% by weight, more usually 4% to 30% by weight and more preferably 6% to 25% by weight, of a cut which is rich in olefins essentially constituted by olefins containing 8 carbon atoms which have been produced by oligomerizing olefins containing 4 (or 4 and 5) carbon atoms. This oligomer cut may also contain non-negligible quantities of paraffins.

The secondary feed may also comprise other oligomers essentially formed from C2 to C10 olefins.

In accordance with the invention, the cracking catalyst is the same for the “maxi propylene” and for the “maxi gasoline” mode. It is a catalyst constituted by an ultra stable Y type zeolite dispersed in an alumina, silica or silica-alumina matrix to which an additive based on ZSM-5 zeolite is added, the total quantity of ZSM-5 crystals in the cracking unit being less than 10% by weight.

Thus, the invention can be defined as a process for the co-production of gasoline and propylene from a hydrocarbon feed with an initial boiling point of more than or equal to 340° C., said process using a catalytic cracking unit followed by an oligomerization unit which can function in accordance with two regimes termed “maxi propylene” (regime 1) and “maxi gasoline” (regime 2) in which:
for the “maxi propylene” regime, the feed for the oligomerization unit is constituted by a C4 cut or a C4/C5 olefinic cut derived from catalytic cracking and the effluents from the oligomerization unit are separated into a C8+ oligomer cut which is recycled at least in part to the inlet to the catalytic cracking unit, the gasoline cut from the process being constituted by the gasoline cut produced in the catalytic cracking unit and the oligomer cut derived from the oligomerization unit which is not recycled for catalytic cracking;
for the “maxi gasoline” regime, the feed for the oligomerization unit is constitute by the olefinic C3/C4 cut derived from catalytic cracking, and the effluents from the oligomerization unit are separated into a C6+ oligomer cut which is added to the gasoline cut derived from catalytic cracking to constitute the gasoline produced by the process;
   the propylene being obtained in the two regimes from FCC effluents after separation in one or more distillation columns.

The catalytic cracking unit may comprise a single reactor processing both the heavy feed and the light feed or two distinct reactors, one processing the heavy feed, the other processing the light feed. Further, each of the reactors may be in riser or dropper mode. Usually, the two reactors will have the same mode of flow.

### A) In the “Maxi Propylene” Regime

1) When the catalytic cracking is carried out in a single reactor in riser mode, the reactor outlet temperature (ROT) is in the range 510° C. to 580° C., preferably in the range 540° C. to 590° C., and the C/O ratio is in the range 8 to 20;
2) When the reactor is in dropper mode, the reactor outlet temperature (ROT) is in the range 550° C. to 590° C. and the C/O ratio is in the range 15 to 50;
3) When the catalytic cracking is carried out in two distinct FCC reactors in dropper mode, the first FCC reactor carrying out cracking of the heavy feed operates at a reactor outlet temperature (ROT1) in the range 510° C. to 580° C., preferably in the range 530° C. to 560° C., and with a C/O ratio in the range 5 to 10, and the second FCC reactor carrying out cracking of C8+ reactors from the oligomerization unit (termed the light feed) operates at a reactor outlet temperature (ROT2) in the range 550° C. to 650° C., preferably in the range 570° C. to 620° C., with a C/O ratio in the range 8 to 25.
4) When the catalytic cracking is carried out in two distinct FCC reactors in dropper mode, the first FCC reactor carrying out cracking of the heavy feed operates at a reactor outlet temperature (ROT1) in the range 550° C. to 700° C. with a C/O ratio in the range 15 to 50, and the second FCC reactor operating cracking of the C8+ oligomers from the oligomerization unit (termed the light feed) operates at a reactor outlet temperature (ROT2) in the range 570° C. to 700° C., with a C/O ratio in the range 15 to 50.

### B) In the “Maxi Gasoline” Regime

1) When the catalytic cracking is carried out in one or two reactors operating in dropper mode, the reactor outlet temperature (ROT) of each cracking reactor is in the range 500° C. to 580° C., preferably in the range 520° C. to 550° C., and the C/O ratio is in the range 5 to 10.
2) When the catalytic cracking is carried out in one or two reactors operating in dropper mode, the reactor outlet temperature (ROT) of each cracking reactor is in the range 530° C. to 650° C., and the C/O ratio is in the range 15 to 25.

The streams of used catalyst derived from the two FCC reactors are separated from the cracking effluents using any gas-solid separation system which is known to the skilled person and regenerated in a common reference zone.

The effluent from the catalytic cracking reactor (or the two effluents if there are two FCC reactors) is sent to a fractionation zone to produce a plurality of cuts including a gasoline cut and a cut containing olefins:
containing 4 (or 4 and 5) carbon atoms for the “maxi propylene” mode;
containing 3 and 4 carbon atoms for the “maxi gasoline” mode.

This cut containing 3 and 4 (denoted C3/C4) or 4 (denoted C4) or 4 and 5 (denoted C4/C5) carbon atoms is sent for oligomerization. It is generally preferable for this cut to undergo selective hydrogenation to reduce the diolefinic compounds and/or acetylenic compounds which may be present in order to increase the oligomerization cycle time. The separation unit constituted by one or more distillation columns is adjusted to allow extraction of the C4 or C4/C5 cut or the C3/C4 cut.

In “maxi gasoline” mode, the major portion or all of the oligomers produced is added to the gasoline cut obtained by fractionation of the catalytic cracking effluent. The gasoline yield, with respect to the quantity of hydrocarbons with a boiling point of more than 340° C., is thus generally in the range 35% to 65% by weight, usually in the range 50% to 60% by weight. The propylene yield with respect to the quantity of hydrocarbons with a boiling point of more than 340° C. is then generally less than 1% by weight and this propylene is in general not specifically recovered.

In “maxi propylene” mode, a portion, i.e. at least 30%, and preferably all of the oligomers produced, is recycled to catalytic cracking. The gasoline yield, with respect to the quantity of hydrocarbons with a boiling point of more than 340° C., is then generally in the range 35% to 55% by weight, usually in the range 40% to 50% by weight. The propylene yield with respect to the quantity of hydrocarbons with a boiling point of more than 340° C. is then generally in the range 4% to 20% by weight, usually in the range 5% to 15% and more usually in the range 7% to 12% by weight.

The particular conditions for the various steps of the process of the invention are described below in more detail.

### 1) Catalytic Cracking (FCC):

The catalyst for the FCC reactor is typically used in the foil of a fine powder with a mean particle diameter which is generally in the range 40 to 140 micrometres, usually in the range 50 to 120 micrometres.

The catalyst for catalytic cracking contains at least one Y type zeolite dispersed in an appropriate matrix such as alumina, silica or silica-alumina.

The catalyst may also comprise at least one zeolite with form selectivity with one of the following structure types: MEL (for example ZSM-11), MFI (for example ZSM-5), NES, EUO, FER, CHA (for example SAPO-34), MFS, MWW, or it may also be one of the following zeolites: NU-85, NU-86, NU-88 and IM-5, which also has form selectivity. The advantage of these zeolites with form selectivity is that better propylene/isobutene selectivity is obtained, i.e. with a propylene/isobutene ratio which is higher in the cracking effluents.

The proportion of zeolite with form selectivity with respect to the total quantity of zeolite may vary as a function of the feeds used and of the desired product structure. Frequently, 2% to 60% is used, preferably 3% to 40% and in particular 3% to 30% by weight of zeolite(s) with form selectivity.

The zeolite or zeolites may be dispersed in a silica, alumina or silica-alumina based matrix, the proportion of zeolite (all zeolites together) with respect to the weight of catalyst usually being in the range 3% to 80% by weight, preferably in the range 4% to 50% by weight, for example in the range 5% to 25% by weight. In the case in which several zeolites are used, they may be incorporated into a single matrix or into a plurality of different matrices. The total amount of zeolite with form selectivity is less than 10% by weight.

The catalyst used in the catalytic cracking reactor may be constituted by an ultra stable Y type zeolite dispersed in an alumina, silica or silica-alumina matrix to which an additive based on ZSM-5 zeolite is added, the total quantity of ZSM-5 crystals being less than 10% by weight.

The unit for separating the catalytic cracking reactor effluents (FCC) generally comprises a primary separation of the FCC effluents, a section for compression and fractionation of gases as well as distillations for fractionation of the various liquid cuts.

This type of fractionation unit is well known to the skilled person and depends on the intended end result. For the “maxi propylene” regime, the C4 cut or the C4/C5 cut is separated from the effluents to send it for oligomerization, and if necessary for selective hydrogenation before oligomerization.

In the “maxi gasoline” regime, the C3/C4 cut is separated from the effluents to send it for oligomerization, and if necessary for selective hydrogenation before oligomerization.

The olefinic cuts from catalytic cracking which are sent for oligomerization may be mixed with olefin-rich cuts imported from other units such as C3/C4 cuts or C4 cuts deriving from a cokefaction unit, steam cracking unit, methanol-olefin conversion unit, etc.

For the C4 cut, isobutene may be extracted, for example by etherification of isobutene by an alcohol, then by distillation, to prevent or limit the presence of isobutene in the oligomerization step, this compound tending to form isomers which can re-crack to isobutene in the FCC, which tends to result in accumulation of this compound if a sufficient isobutene purge is not carried out.

After extracting the isobutene, it is also possible to carry out extractive distillation, for example with a solvent which may be N-methyl pyrrolidone (NMP) or dimethylsulphoxide (DMSO) or an isomer thereof, to extract the unsaturated fraction from the paraffins in the feed which are then mixed with the solvent. This means that a mixture of butenes (or even butenes/pentenes) or propylene/butenes freed of C4 (or even C4/C5) or C3/C4 paraffins can be recycled to the selective hydrogenation or oligomerization.

### 2) Selective Hydrogenation (Optional)

The C4 or C4/C5 cut or the C3/C4 cut of the FCC effluents contains a small quantity of dienes (diolefins) and acetylenes which increase coking of the oligomerization catalyst and thus reduce the cycle time of the oligomerization reactor. Selective hydrogenation of dienes and acetylenes to mono-olefins is thus preferable in the process of the invention, although it is not vital.

The principal aim of this step is to transform the diolefins (or dienes) into mono-olefins. The mono-olefins are the source of the oligomers produced in oligomerization step 3. Another aim of this step is to eliminate traces of acetylenic hydrocarbons present in these cuts since they are unwanted compounds as regards oligomerization. The acetylenic compounds are also transformed into mono-olefins. The residual quantity of diolefins+acetylenics of the selective hydrogenation effluent is typically less than approximately 1000 ppm by weight, preferably less than 100 ppm by weight, and more preferably less than 20 ppm by weight.

In general, this selective hydrogenation step is carried out using a catalyst comprising at least one metal selected from the group formed by nickel, palladium and platinum, deposited on a support comprising alumina, silica or silica-alumina.

The quantity of palladium on the support may typically be 0.01% to 5% by weight, preferably 0.05% to 1% by weight.

The operating temperature for the selective hydrogenation is generally in the range 0° C. to 200° C., the pressure typically is in the range 0.1 to 5 MPa, usually in the range 0.5 to 5 MPa, the hourly space velocity is typically in the range 0.5 to 20 m³ per hour per m³ of catalyst, preferably in the range 0.5 to 5 m³ per hour per m³ of catalyst, and the H₂/(acetylenic+diolefinic compounds) molar ratio is generally in the range 0.5 to 5, preferably in the range 1 to 3.

In order to carry out selective hydrogenation, one or more fixed bed reactors is used, in downflow co-current mode for the feed to be processed and the hydrogen (or a gas containing a substantial molar fraction of hydrogen, for example at least 50%), or in downflow mode for the feed to be processed and upflow mode for the hydrogen (or hydrogen-rich gas).

When the C5 cut is sent for oligomerization, it is possible to selectively hydrogenate this cut in a unit which is separate from the unit for hydrogenation of the C3 and C4 cuts, such as in a selective gasoline hydrogenation unit, for example.

### 3) Oligomerization

The aim of this step is to oligomerize C4 or C4/C5 olefins (“maxi propylene” regime) or C3/C4 olefins (“maxi gasoline” regime) to obtain a mixture of hydrocarbons containing mono-olefins with a number of carbon atoms equal to eight or more. Typically, starting from C4 olefins, oligomers are obtained which mainly contain 30 or fewer carbon atoms, primarily in the range 8 to 20 carbon atoms.

Oligomerization is distinguished from polymerization by adding a limited number of molecules, the number of added molecules in the context of the invention being in the range 2 to 10, limits included, and generally in the range 2 to 5, in particular in the range 2 to 4.

The oligomers may, however, include traces of olefins which have been oligomerized with a number of molecules exceeding 10. Usually, these traces represent less than 5% by weight with respect to the oligomers fowled.

Oligomerization may be carried out in one or more steps, with one or more reactors and one or more catalysts. The following description of the catalyst and the operating conditions may be applied to any one of the steps and/or any one of the reactors:
in “maxi propylene” regime, the oligomerization catalyst used is preferably a catalyst based on silica-alumina. The temperature is in the range 100° C. to 200° C., and preferably in the range 140° C. to 160° C. The pressure is in the range 0.5 to 10 MPa.
In “maxi gasoline” regime, the oligomerization catalyst used is preferably a catalyst based on silica-alumina. The operating temperature is in the range 100° C. to 200° C., preferably in the range 140° C. to 160° C. The operating pressure is in the range 2 to 10 MPa (1 MPa=10⁶ pascal).

The invention will now be explained in more detail with the aid of the description of FIGS. 1 and 2.

FIG. 1 shows the facility for carrying out the process of the invention in the “maxi propylene” operating regime.

The vacuum distillate or atmospheric residue feed is introduced via line 1.

The catalytic cracking reactor (R1) is supplied via two separate lines 1 and 9.

The effluents from the catalytic cracking (R1) are evacuated via line 2 and introduced into a separation zone S1.

The separation zone S1 typically comprises a gas compressor and distillation/absorption means.

The C3+C4 cut of the effluents from the catalytic cracking reactor (R1) is sent to the separation zone S2 via line 4. The gasoline corresponding to a C5-220° C. cut is evacuated via line 15, and the other FCC effluents are evacuated via line 3.

The separation zone S2 typically comprises distillation means. The C3 cut containing mainly propylene is evacuated via line 5. The highly olefinic C4 cut is sent to the oligomerization reactor R2 via line 6.

The oligomerization effluents are extracted via line 7 and introduced into a separation zone S3. The separation zone S3 typically comprises distillation of the oligomerization effluents to recover the heavier oligomers, and the unreacted C4 cut. The C4 cut is mainly constituted by paraffinic compounds and a minority of untransformed olefinic compounds. This C4 cut is evacuated via line 8.

The C8+ oligomers are partially and preferably completely introduced into the catalytic cracking reactor R1 via line 9, the line 14 corresponding to oligomers which are not recycled to R1. In the case in which the oligomers are not all sent for catalytic cracking, the major portion of the remaining oligomers are mixed with the gasoline from catalytic cracking. Cracking the oligomers in R1 means that the propylene yield of the facility can be increased.

FIG. 2 shows the facility for carrying out the process of the invention in “maxi gasoline” regime.

The catalytic cracking reactor R1 is supplied with vacuum distillate or atmospheric residue via line 1.

The C3+C4 cut from the FCC effluents is sent to the oligomerization reactor R2 via line 10.

The C3+C4 cut which has not reacted in R2 is sent to the separation zone S2 via line 12. The C4 cut, separated from the C3 cut in S2, is evacuated via line 13.

The C6+ oligomers formed are not recycled and form a gasoline cut (line 11) which joins up with the gasoline cut from the catalytic cracking reactor (line 15). The gasoline produced by the process is thus constituted by a combination of effluents from lines 15 and 11.

### EXAMPLES

Three examples will now be described to illustrate the improved flexibility of the process over prior art processes.

### Example 1

### Prior Art

An atmospheric residue feed with the principal characteristics given below was processed in a FCC type catalytic cracking unit operating in two regimes (“maxi gasoline” and “maxi propylene”):

| | |
|---|---|
| Density | 0.93 |
| Viscosity at 50° C. (cSt) | 84 |
| Conradson carbon | 4.36 |
| Hydrogen (wt %) | 12.3 |
| TBP 10% (° C.) | 387 |
| TBP 90% (° C.) | 723 |

The catalyst had the following characteristics:
catalyst: Y zeolite dispersed in a matrix based on silica-alumina;
mean catalyst diameter: 70 micrometres;
density of grain: 1250 kg/m³.

The pressure in the reaction zone was equal to 0.2 MPa and the operating conditions for the “maxi propylene” and “maxi gasoline” regimes were as follows:

| Mode | “Maxi propylene” | “Maxi gasoline” |
|---|---|---|
| Reactor outlet | 550 | 528 |
| temperature (° C.) | | |
| C/O ratio (by weight) | 7.5 | 7.1 |

The yields of propylene, C4 cut and gasoline with respect to the feed were as follows for the regimes:

| Yield (% by weight of | | |
|---|---|---|
| fresh feed) | “Maxi propylene” | “Maxi gasoline” |
| Propylene | 7.2 | 4.8 |
| C4 cut | 13.7 | 9.8 |
| Gasoline (C5-220° C.) | 44.1 | 47.1 |

### Example 2

### In Accordance with the Invention, Illustrated in FIG. 1

The atmospheric residue processed in Example 1 was used in the process of the invention in “maxi propylene” mode under the atmospheric residue cracking conditions of Example 1 for “maxi propylene” mode (C/O=7.5 and 550° C. at the reactor outlet).

The C4 cut of the catalytic cracking effluent was separated in the separation zone S1 then S2, then introduced into the oligomerization reactor R2 which operated under the following conditions:
pressure: 6.0 MPa;
temperature: 140-160° C.;
space velocity: 0.5 to 1

The oligomerization catalyst was amorphous silica-alumina.

Approximately 83% by weight of C4 olefins were principally oligomerized to C8 olefins.

The oligomers, separated from the unreacted oligomers and C4 paraffins in the separation zone S3, were completely recycled to the catalytic cracking reactor R1.

Cracking of these oligomers could increase the propylene yield and the non-cracked oligomers increased the overall gasoline yield.

The yields of propylene, C4 cut and gasoline under the “maxi propylene” regime with respect to the atmospheric residue feed were as follows:

| Yield (% by weight of | Invention, in accordance with |
|---|---|
| fresh feed) | FIG. 1 |
| Propylene | 9.3 |
| C4 cut | 5.9 |
| Gasoline (C5-220° C.) | 49.0 |

Thus, compared with the “maxi propylene” regime of the prior art, there was an increase of 2 points (9.3−7.2) for the propylene and at the same time 5 points (49−44.1) for the gasoline.

### Example 3

### In Accordance with the Invention, Illustrated in FIG. 2

Again, the atmospheric residue processed in Example 1 was used in the process of the invention in “maxi gasoline” mode under the atmospheric residue cracking operating conditions of Example 1 for “maxi gasoline” mode (C/O=7.1 and 528° C. at the reactor outlet).

The C3+C4 cut separated in the separation zone S1 was introduced into the oligomerization reactor R2 which operated under the following conditions:
pressure: 6.6 MPa;

temperature: 130-160° C.;
space velocity: 0.5 to 1.

Again, the oligomerization catalyst was amorphous silica-alumina.

Approximately 87% by weight of C3+C4 olefins were oligomerized to C8, C9 and C12 olefins.

The oligomers, separated from the unreacted oligomers and paraffins in the separation zone S3, were added to gasoline from cracking of atmospheric residue and in a very small proportion to the gas oil pool.

The yields of propylene, C4 cut and gasoline in “maxi gasoline” regime (illustrated in FIG. 2) with respect to the atmospheric residue feed were as follows:

| Yield (% by weight of | Invention, in accordance with |
|---|---|
| fresh feed) | FIG. 2 |
| Propylene | 0.2 |
| C4 cut | 4.2 |
| Gasoline (C5-220° C.) | 55.2 |

The gasoline yield was increased by 8 points (55.2−47.1) compared with the prior art “maxi gasoline” regime.

1. A process for the co-production of gasoline and propylene from a heavy hydrocarbon feed with an initial boiling point of more than 340° C. using a catalytic cracking unit followed by an oligomerization unit which can function in accordance with two regimes termed “maxi propylene” and “maxi gasoline”, in which:
for the “maxi propylene” regime, the feed for the oligomerization unit is constituted by the C4 cut or the C4/C5 olefinic cut derived from catalytic cracking and the effluents from the oligomerization unit are separated into a C8+ oligomer cut which is recycled at least in part to the inlet to the catalytic cracking unit, the gasoline cut from the process being constituted by the gasoline cut produced in the catalytic cracking unit and the oligomer cut derived from the oligomerization unit which is not recycled for catalytic cracking;
for the “maxi gasoline” regime, the feed for the oligomerization unit is constituted by the olefinic C3/C4 cut derived from catalytic cracking, and the effluents from the oligomerization unit are separated into a C6+ oligomer cut which is added to the gasoline cut derived from catalytic cracking to constitute the gasoline produced by the process;
the propylene being obtained in the two regimes from FCC effluents after separation in one or more distillation columns.

2. A process for the co-production of gasoline and propylene from a hydrocarbon feed with an initial boiling point of more than 340° C. according to claim 1, in which under the “maxi propylene” regime, when the catalytic cracking is carried out in a single reactor in riser mode, the reactor outlet temperature (ROT) is in the range 510° C. to 580° C., preferably in the range 540° C. to 590° C., and the C/O ratio is in the range 8 to 20.

3. A process for the co-production of gasoline and propylene from a hydrocarbon feed with an initial boiling point of more than 340° C. according to claim 1, in which in the “maxi propylene” regime, when catalytic cracking is carried out in two distinct FCC reactors in riser mode, the first FCC reactor carrying out cracking of the heavy feed operates at a reactor outlet temperature (ROT1) in the range 510° C. to 580° C., preferably in the range 530° C. to 560° C., and with a C/O ratio in the range 5 to 10, and the second FCC reactor carrying out cracking of C8+ oligomers derived from the oligomerization unit (termed the light feed) operates at a reactor outlet temperature (ROT2) in the range 550° C. to 650° C., preferably in the range 570° C. to 620° C., with a C/O ratio in the range 8 to 25.

4. A process for the co-production of gasoline and propylene from a hydrocarbon feed with an initial boiling point of more than 340° C. according to claim 1, in which in the “maxi gasoline” regime, when catalytic cracking is carried out in one or two reactors operating in riser mode, the reactor outlet temperature (ROT) is in the range 500° C. to 580° C., preferably in the range 520° C. to 550° C., and the C/O ratio is in the range 5 to 10.

5. A process for the co-production of gasoline and propylene from a hydrocarbon feed with an initial boiling point of more than 340° C. according to claim 1, in which in the “maxi propylene” regime, when catalytic cracking is carried out in a single reactor in riser mode, the reactor outlet temperature (ROT) is in the range 550° C. to 700° C., and the C/O ratio is in the range 15 to 50.

6. A process for the co-production of gasoline and propylene from a hydrocarbon feed with an initial boiling point of more than 340° C. according to claim 1, in which in the “maxi propylene” regime, when catalytic cracking is carried out in two distinct FCC reactors in dropper mode, the first FCC reactor carrying out cracking of the heavy feed operates at a reactor outlet temperature (ROT1) in the range 550° C. to 700° C., and a C/O ratio in the range 15 to 50, and the second FCC reactor carrying out cracking of C8+ oligomers derived from the oligomerization unit (termed the light feed) operates at a reactor outlet temperature (ROT2) in the range 570° C. to 700° C., with a C/O ratio in the range 15 to 50.

7. A process for the co-production of gasoline and propylene from a hydrocarbon feed with an initial boiling point of more than 340° C. according to claim 1, in which in the “maxi gasoline” regime, when catalytic cracking is carried out in one or two reactors operating in dropper mode, the reactor outlet temperature (ROT) is in the range 530° C. to 650° C. and the C/O ratio is in the range 15 to 25.

8. A process for the co-production of gasoline and propylene from a hydrocarbon feed with an initial boiling point of more than 340° C. according to claim 1, in which in the “maxi propylene” regime, the oligomerization unit operates at a temperature in the range 100° C. to 200° C., preferably in the range 140° C. to 160° C., and at a pressure in the range 0.5 to 10 MPa.

9. A process for the co-production of gasoline and propylene from a hydrocarbon feed with an initial boiling point of more than 340° C. according to claim 1, in which in the “maxi gasoline” regime, the oligomerization unit operates at a temperature in the range 100° C. to 200° C., preferably in the range 140° C. to 160° C., and at a pressure in the range 2 to 10 MPa.

10. A process for the co-production of gasoline and propylene from a hydrocarbon feed with an initial boiling point of more than 340° C. according to claim 1, in which the C4 cut or the C4/C5 cut or the C3/C4 cut is sent to a selective hydrogenation unit located upstream of the oligomerization unit, said selective hydrogenation unit operating at a temperature in the range 0° C. to 200° C., a pressure in the range 0.5 to 5 MPa, a space velocity in the range 0.5 to 5 m³ per hour and per m³ of catalyst, with the H₂/(acetylenic+diolefinic compounds) molar ratio being in the range 1 to 3.

11. A process for the co-production of gasoline and propylene from a hydrocarbon feed with an initial boiling point of more than 340° C. according to claim 1, in which the catalyst used in the catalytic cracking reactor is constituted by an ultra stable Y type zeolite dispersed in an alumina, silica, or silica-alumina matrix, to which an additive based on ZSM-5 zeolite is added, the total quantity of ZSM-5 crystals being less than 10% by weight.
